(19) United States
(12) Patent Application Publication
(10) Pub. No.: US 2003/0055468 A1
(43) Pub. Date: Mar. 20, 2003

(54) Bi-lateral cervico-facial stimulation system

(76) Inventor: Michael Sachs, New York, NY (US)

Correspondence Address: FITZPATRICK CELLA HARPER & SCINTO, 30 ROCKEFELLER PLAZA, NEW YORK, NY, 10112, US

(21) Appl. No.: 09/954,070

(22) Filed: Sep. 18, 2001

(51) Int. Cl. A61N 1/18

(52) U.S. Cl. 607/48

(57) ABSTRACT

A device is provided which senses muscular action in one portion of the body, particularly on an uninjured side of the face, preferably by sensing nerve impulses that produce that action, and in response to such sensing generates an electrical signal to stimulate a similar action in a muscle elsewhere in the body, such as the corresponding muscle on the other side of the face.

### BACKGROUND OF THE INVENTION

1. Field of the Invention

The present invention relates generally to the fields of surgical implants and of devices for use in treating injuries resulting from accidents or the like, and more particularly relates to a device by means of which a patient suffering from loss of muscular control on one side of the body, such as the face, due to nerve injury, can be treated, such that the patient is able to produce bilaterally symmetric muscular activity, especially facial expressions, in spite of the injury.

2. Related Background Art

In certain injuries, damage to nerves may result in the permanent loss of control of one or more muscles. When the affected muscles are in the face, this type of injury is especially noticeable, and impairs the normal use of the face for expression. Consequently, this type of injury can have severe emotional effects in addition to the physical ones.

It would be desirable to have a way of treating such injuries that would restore to the patient the ability to use the muscles on both sides of the body. To date, so far as the present inventor is aware, no such treatment exists.

### SUMMARY OF THE INVENTION

Accordingly, it is an object of the invention to provide a device suitable for use in such a treatment.

According to one aspect of the invention, a device is provided which senses muscular action in one portion of the body, particularly on the uninjured side of the face, preferably by sensing the nerve impulses that produce that action, and in response to such sensing generates an electrical signal to stimulate a similar action in a muscle elsewhere in the body, preferably the corresponding muscle on the other side of the body. (Throughout this description, the term “corresponding” means the nerve or muscle on the other side of the body, having the same anatomical name.) Preferably, the main portion of the device is implanted in the mastoid bone, although implantation elsewhere is also within the scope of the invention, as is locating the main portion of the device external to the patient's body.

If appropriate, the device may include several detectors or sensors, the number being equal to the number of muscles whose use is impaired by nerve injury.

### BRIEF DESCRIPTION OF THE DRAWINGS

FIG. 1 is a schematic diagram showing the major components of a device according to one preferred embodiment of the present invention.

FIG. 2 is a view illustrating some of the musculature of the face, indicating the detection of muscular action in the right side of the patient's face, the sending of impulses indicating that action to the main portion of the device of the invention, and the generation of signals to electrodes in the left side of the face to stimulate similar muscular action.

FIG. 3 shows additional details of the embodiment of FIG. 1.

### DETAILED DESCRIPTION OF THE PREFERRED EMBODIMENTS

The preferred embodiments of the present invention described herein are for use in treating an injury to the face. This is merely by way of example, however, and the device of the invention can be used in treating any portion of the body desired, where injury has occurred to one side that results in loss of control of one or more muscles on that side.

In particular, as has been pointed out to the inventor by Dr. Charles Kimmelman, an injury that results in the loss of control of muscle tissue on one side of the larynx, which by affecting the speech may have emotional effects on a patient similar in severity to those of a facial injury, can be treated using a device constructed according to the present invention. Thus, throughout the following description, it should be kept in mind that the devices being described can be used to treat laryngeal and other non-facial injuries.

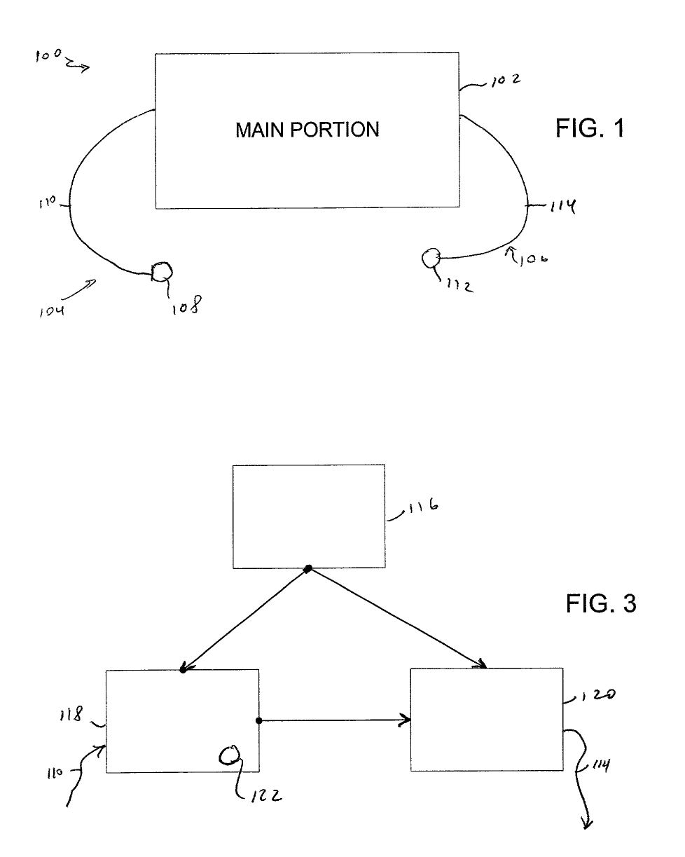

As shown in FIG. 1, the first preferred embodiment 100 of the present invention has a main body portion 102, a sensor unit 104, which receives or senses a nerve impulse from the patient's body, and a nerve stimulating unit 106, which delivers a stimulating signal to another portion of the patient's body. The sensor unit 104 comprises at least one sensor electrode 108 and an electrical lead 110 that conveys electrical signals detected by the sensor electrode 108 to the main body portion 102. The nerve stimulating unit 106 contains at least one stimulating electrode 112 and a lead 114 connecting that electrode to the main body portion 102. (For simplicity, this embodiment will be described in terms of a single sensor electrode and a single stimulating electrode.)

In this embodiment, the main body portion 102 contains a number of components (see FIG. 3), including a power source 116 (preferably a long-lived battery such as is commonly used to power cardiac pacemakers; the battery may be of a design to permit recharging by radio waves), a circuit 118 adapted to recognize when a signal has been sensed by the sensor electrode (“recognition circuit”), and a signal generator 120.

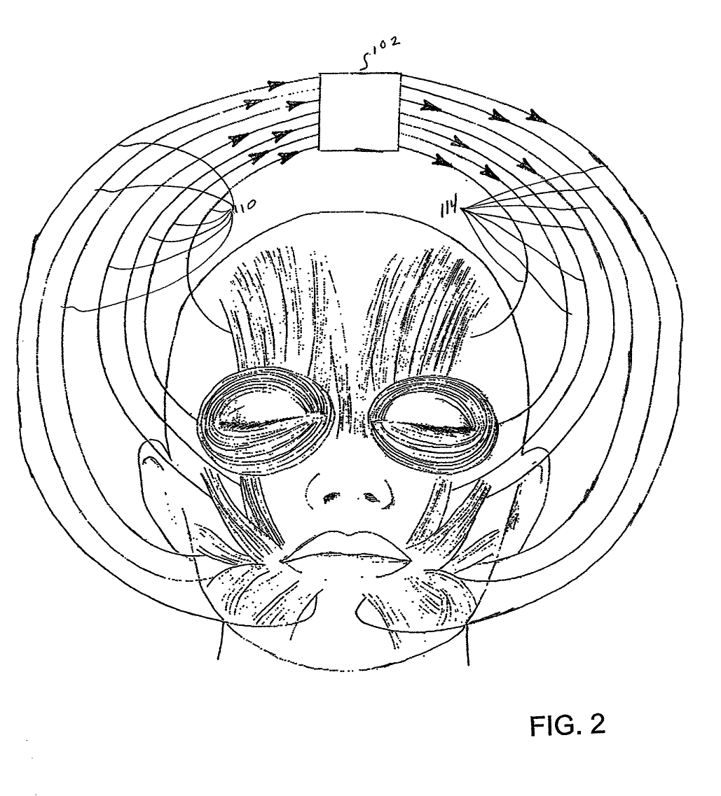

The stimulating electrode 112 is located in the vicinity of an injured nerve on one side of the patient's face (this is illustrated schematically in FIG. 2, although with leads for seven sensor electrodes and for seven stimulating electrodes shown by way of example). The nerve may be one that has been damaged or severed as a result of an accident or the like, and the electrode is placed adjacent an uninjured portion of the nerve, which portion is still capable of transmitting electrical impulses on to the muscle tissue served by that nerve. Alternatively, the stimulating electrode may be placed into the muscle, instead of the nerve, or it may be located in both types of tissue.

Similarly, the sensor electrode is located adjacent the uninjured corresponding nerve on the other side of the patient's face (by “adjacent” is meant simply that the sensor electrode is to be positioned where it can detect an impulse transmitted through the uninjured nerve; provided such detection is possible, the exact location of the sensor electrode can be varied without departing from the scope of the present invention). The main body portion 102 of the device may be surgically implanted in the patient's body, for example, in the mastoid bone.

In use, a nerve impulse transmitted by the nerve to which the sensor electrode 108 is adjacent, produces a signal in that electrode. That signal is conveyed by the electrical lead 110 to the recognition unit 118 in the main body portion of the device. The recognition unit 118 tests the signal from electrical lead 110 to ascertain that the signal is indeed a result of the transmission of an impulse in the nerve of interest, and is not spurious. This testing may simply be a comparison of the signal to a threshold voltage value, but if desired, a more sophisticated test may be applied. For example, it is within the scope of the present invention to compare the waveform of the signal received by the sensor electrode to one or more stored waveforms, and to accept the signal as genuine if it matches any of the stored waveforms to some predefined level of accuracy.

If the received signal is accepted as indicating the generation of a nerve impulse, the recognition unit outputs a signal indicating this fact to the signal generator, which in response generates a signal to the stimulating electrode 112. This signal is applied, via the stimulating electrode 112, to the injured nerve, at a location from which the nerve is able to transmit the signal to the muscle tissue served by that nerve. (Alternatively, this signal may be delivered directly into the muscle tissue, or into both the nerve and muscle.) This muscle tissue responds by contracting as it would do if stimulated by a natural nerve impulse transmitted by an uninjured nerve. Thus, when a nerve impulse produces a muscular action on the uninjured side of the face, a similar muscular action is produced artificially in the corresponding muscle on the injured side of the face.

The signal generator 120, in its simplest form, may simply consist of an on-off switch (not shown) that, upon actuation, closes a circuit to apply a predetermined voltage to the stimulating electrode lead 114. In this construction, the recognition unit 118 continues outputting its signal to the signal generator 120 as long as the signal being received from the sensor electrode lead 108 continues to pass the thresholding test by which it is recognized. As a result, the muscle tissue on the injured side of the face continues to receive a stimulus as long as the uninjured nerve on the other side of the face is transmitting a signal, resulting in a symmetrical muscular action on the two sides of the patient's face.

In another embodiment, the signal generator 120 may produce a train of pulses of predetermined height (voltage), width and spacing, as long as the recognition unit continues to receive a valid signal from the sensor electrode. (By “valid” is meant that the signal passes the test to which the recognition unit subjects it.) In either approach (one pulse or pulse train), however, it is also within the scope of the invention to generate a signal whose waveform more closely approximates that of a natural nerve impulse, using well-known wave-shaping techniques.

Preferably, the recognition unit 118 includes a calibration control (indicated schematically at 122), by means of which the exact value of the threshold can be adjusted at the time of implantation of the device; still more preferably, the calibration control also permits re-adjustment of the threshold subsequent to implantation of the device (for example, using a radio signal, or otherwise). By means of making this adjustment, the surgeon can adjust the sensitivity of the recognition unit to compensate for the exact distance of the sensor electrode 108 from the nerve. One manner of doing so may be for the surgeon to stimulate one or a series of impulses in that nerve, each of predetermined voltage and pulse-width, while adjusting the threshold. (This can be done using known nerve stimulators.) Knowing the strength of the impulse(s) being applied to the nerve, the surgeon can determine when the threshold is at an appropriate level for the exact actual location of the sensor electrode 108 relative to the nerve.

Many additional variations of the construction of this device are possible within the scope of the invention. For example, the above-described functions of the recognition unit 118 and of the signal generator 120 may be performed in part by a microprocessor instead of by analog or specially-designed digital circuitry. Again, while it is especially contemplated to detect nerve impulses traversing the healthy nerve on the uninjured side of the body, it is within the scope of the invention to detect the resulting muscular action, instead of or in addition to detecting the nerve impulse. Moreover, as stated above, it is also within the scope of the invention to apply the stimulus directly to muscle tissue, instead of or in addition to applying it to nerve tissue.

Again, if several facial nerves have sustained injury, the device may include at least a second sensor, located to detect action of second muscle tissue on the uninjured side of the face (see FIG. 2, with seven sensor electrodes provided for). In this case, plural leads 114 are provided connecting the signal generator 120 to respective portions of muscle tissue in the second side of the face, and the main body portion 102 of the device includes a selector (not shown) that determines on which of the leads 114 the signal is output. Similarly, the recognition unit 118 has means of determining or identifying which of the sensor electrodes is producing the signal being input to the recognition unit 118. To permit the simultaneous detection and duplication of several nerve impulses, the main portion 102 of the device as shown in FIG. 2 preferably has plural separate recognition circuits, one for each sensor electrode, and an equal number of signal generators. While this arrangement is particularly simple, other approaches, which will be readily apparent to those of ordinary skill in circuit design, are also within the scope of the invention.

Again, while it is particularly contemplated that the power source 116 be a long-lived battery, any power source that meets the size, weight and longevity requirements in a particular case can be used, and is within the scope of the invention.

Moreover, while the present invention has been described in connection with facial injuries, the invention is applicable to any situation in which it is desired to apply stimulation to one portion of the body in response to detection of nerve activity or muscular activity in another portion of the body.

All the above-described circuitry and functions can be implemented using either off-the-shelf components, or components and designs that are well within the ordinary level of skill of those in the relevant arts.

In addition, many additional features and variations of the present invention will now be apparent to those of ordinary skill. Accordingly, the scope of the present invention is to be determined solely by the terms of the claims appended hereto, and not by the details of the preferred embodiments described above.

What is claimed is:

1. A device for inducing muscular action in the body of a patient, said device comprising:
a sensor, located to detect action of muscle tissue in one portion of the body; and
a signal generator connected to issue a signal in response to detection by said sensor, said signal generator being connected to issue the signal to a location such as to cause corresponding muscle tissue in a second portion of the body to contract.

2. A device according to claim 1, wherein the first portion of the body is one side of the face, and the second portion of the body is a second side of the face.

3. A device according to claim 2, wherein said sensor senses muscular action by sensing a nerve impulse being transmitted to the muscle tissue in the one side of the face.

4. A device according to claim 2, wherein said sensor directly senses muscular action of the muscle tissue in the one side of the face.

5. A device according to claim 2, further comprising at least a second sensor, located to detect action of second muscle tissue, plural conductors connecting said signal generator to respective portions of muscle tissue in the second side of the face, and a selector arranged to determine on which of said plural conductors the signal is output.

6. A device according to claim 2, further comprising a power source.

7. A device according to claim 2, further comprising a housing containing said signal generator and said power source.

8. A device according to claim 7, wherein said housing is sufficiently small for implantation into the patient's mastoid bone.

9. A device according to claim 6, wherein said power source comprises a battery.

10. A method for providing inducing muscular action in the body of a patient, comprising the steps of:
detecting action of muscle tissue in one portion of the body; and
in response to detection in said detecting step, generating a signal and communicating the signal to a location such as to cause corresponding muscle tissue in a second portion of the body to contract.

11. A method according to claim 10, wherein the first portion of the body is one side of the face, and the second portion of the body is a second side of the face.

12. A method according to claim 11, wherein said detecting step includes detecting a nerve impulse being transmitted to the muscle tissue in the one side of the face.

13. A method according to claim 11, wherein said detecting step includes directly detecting the muscular action of the muscle tissue in the one side of the face.

14. A method according to claim 11, wherein said detecting step is performed to detect muscular action in progress or about to occur in any one of plural muscles on the one side of the face, and wherein said generating step is performed so as to communicate the signal selectively to that muscle tissue on the second side of the face which corresponds to the one muscle.
